(19) United States
(12) Patent Application Publication
(10) Pub. No.: US 2010/0018861 A1
(43) Pub. Date: Jan. 28, 2010

(54) ELECTROMOTIVE LIQUID HANDLING METHOD AND APPARATUS

(75) Inventors: Igor Mezic, Goleta, CA (US); Frederic Bottausci, Santa Barbara, CA (US)

Correspondence Address: GATES & COOPER LLP;HOWARD HUGHES CENTER, 6701 CENTER DRIVE WEST, SUITE 1050, LOS ANGELES, CA, 90045, US

(73) Assignee: THE REGENTS OF THE UNIVERSITY OF CALIFORNIA, Oakland, CA (US)

(21) Appl. No.: 12/056,113

(22) Filed: Mar. 26, 2008

Related U.S. Application Data
(60) Provisional application No. 60/908,112, filed on Mar. 26, 2007.

(51) Int. Cl. C07K 1/26; G01N 27/26

(52) U.S. Cl. 204/450; 204/600; 204/643

(57) ABSTRACT

An apparatus and method to achieve manipulation of particles and/or solutions through the induction of electromagnetic fields inside liquid contained inside vessels are disclosed. A “vessel” denotes specifically either a microtitter plate (microplate or well-plate, 1536, 3456 or any other format) well or hybridization solution placed on microarrays for hybridization experiment purposes or more generally any volume containing liquid solution. The manipulation is performed by bringing the active part of the device into contact with the fluidic solution, wherein electromagnetic fields and/or induced fluid flow are used to perform specific manipulations including transport, separation, concentration, mixing, reaction and electroporation. The invention can be used to enhance processing in a number of standard assays and protocols, including the Polymerase Chain Reaction, kinase-based assays, ELISA assays and electroporation.

### CROSS-REFERENCE TO RELATED APPLICATION

This application claims the benefit under 35 U.S.C. Section 119(e) of the following co-pending and commonly-assigned U.S. patent application:

U.S. Provisional Application Ser. No. 60/908,112, filed on Mar. 26, 2007, by Igor Mezic and Frederic Bottausci, entitled “ELECTROMOTIVE LIQUID HANDLING METHOD, APPARATUS, AND PROCESS,” attorneys' docket number 30794.224-US-P1 (2007-425-1);

which application is incorporated by reference herein.

### STATEMENT REGARDING FEDERALLY SPONSORED RESEARCH AND DEVELOPMENT

This invention was made with Government support under Grant No. DMS-0507256 awarded by the NSF. The Government has certain rights in this invention.

### BACKGROUND OF THE INVENTION

1. Field of the Invention

The present invention relates in general to the field of manipulating fluid flow and/or particle motivating force and is related to separation, concentration, transport, dispersion, reaction and mixing apparatus, method and process. More particularly, the present invention relates to improved manipulation by bringing the present invention in contact with the solution. Moreover, the invention can be embedded into existing liquid containing vessels such as well-plates and microarrays.

2. Description of the Related Art

(Note: This application references a number of different publications as indicated throughout the specification by one or more reference numbers within brackets, e.g., [x]. A list of these different publications ordered according to these reference numbers can be found below in the section entitled “References.” Each of these publications is incorporated by reference herein.)

Devices using electrokinetic properties (electrophoresis, dielectrophoresis, electroosmosis and electrothermal convection) have been used to manipulate fluids and particles.

Electrophoresis is a technique for manipulating components of a mixture of charged molecules (proteins, DNAs, or RNAs) in an electric field within a gel or other support. Under AC electric field, uncharged particles suspended in a dielectric media can be polarized and further manipulated. If the field is spatially inhomogeneous, it exerts a net force on the polarized particle known as dielectrophoretic (DEP) force[1]. This force depends upon the temporal frequency and spatial configuration of the field as well as on the dielectric properties of both the medium and the particles. Single frequency electric fields can be used to transport and separate particles.

Fluid motion can also be induced by applying an electric field onto a solution. The force driving the fluid thus originates in the bulk (buoyancy, electrothermal effect) or at the interface between the fluid and the device containing the fluid (electroosmosis).

The buoyancy generates a flow because of a density gradient. It can be produced by internal or external heating. An electric field is often used as internal energy source. Applied to a solution, part of the electric energy dissipates in the fluid by Joule effect and locally heats the fluid. Furthermore, local heating creates gradients of conductivity and permittivity. The fluid can then move under the influence of an electrothermal flow [2, 3, 4].

Under certain conditions (material properties, conductivity and permeability of the fluid and the device containing the fluid), ion layers develop at the fluid-surface interface due to chemical associations or dissociations and physical adsorption on or desorption from the solid surface. Ion layers can also be generated at the surface of electrodes where a potential is externally imposed. Applying an electric field with a tangential component to the layers moves the ions which carry the fluid along by viscous force. This process produces a bulk flow [2, 3, 4].

Coupled with an electrohydrodynamic flow, several electrode geometries have been designed as a tool to manipulate fluids and particles. Interdigitated castellated electrodes are, for instance, designed to trap and separate particles [5, 6]. Polynomial electrodes [7], planar electrodes [8, 9], quadripolar electrodes [27] or more complex geometries [10] have also been proposed.

Micro Technology Applied to Biological Problems

Massively parallel hybridization [1]-13] improves the way many biological and medical analyses are performed both in research and clinical applications, but there is still a lack of an efficient multipurpose device. As sample volumes used in massive parallel systems become smaller and smaller (micro- to nanoliter or even smaller) it is more challenging to manipulate the fluids since the fluid viscosity dominates any convection. Multiple reports have shown that micromixing, transport or concentration improves hybridization reaction [14-16, 17, 18]. Micromixing can be achieved by ultrasonic agitation (the nucleation of bubbles creates small jets that enhance the mixing) [19] or by vortexing or agitating the solution and creating convection [20]. Micromixing can also be produced by surface wave generation [21] for instance.

What is needed then are improved methods, processes and general apparatus to efficiently and accurately mix, separate, concentrate, and transport small volume of fluids with or without particles (e.g., atoms, molecules, cells in biological and chemical assays) using combined fluid flow and/or electrokinetic methods. The present invention satisfies that need.

### SUMMARY OF THE INVENTION

The present invention discloses an apparatus and method to achieve manipulation of particles and/or solutions. The invention uses electrokinetic properties. The manipulation is performed by bringing the active part of the device into contact with the fluidic solution. For the purpose of this document, a “vessel” will denote specifically either a microtitter plate (microplate or well-plate) well or hybridization solution placed on microarrays for hybridization experiment purposes or more generally any volume containing liquid solution. The electrodes (made of any applicable material) are inserted inside one or more vessels containing one or more fluids or one or more fluids and one or more types of particles for the purpose of manipulating fluid(s) and/or particles. The manipulations can include concentration, separation, transport, mixing or cell electroporation.

A device containing electrodes capable of inducing electrokinetic (including electroosmotic and electrothermal) fluid flow inside vessels (including microplates or well-plates). The device is either applied externally by inserting electrodes inside the vessel, or the device is built into the vessel, itself, if flow is generated by forces other than single-frequency electroosmosis. The device can be used for general manipulation of fluids and particles inside the vessel, including concentration, separation, transport, mixing or cell electroporation. The device is tunable, so that by applying different DC and/or AC voltages, different flow effects can be induced and adapted to efficiently manipulate the fluids and particles contained inside the vessel. The device can perform one or more particle manipulation operations.

A method where more than one frequency of AC field is used to induce fluid flows sequentially in time or simultaneously to induce fluid flow and electromagnetic field for the purpose of manipulating particles (including concentration, separation, transport, mixing of the particles or cell electroporation). The flow and electromagnetic field can be applied by electrodes built into the device (thus being an integral part of the device) or applied externally.

### BRIEF DESCRIPTION OF THE DRAWINGS

Referring now to the drawings in which like reference numbers represent corresponding parts throughout:

FIGS. 1(a)-1(e) are block diagrams that illustrate some examples of the arrangement of electrode arrays used in an embodiment of the present invention, wherein FIG. 1(a) is a drawing of a planar electrode array, FIG. 1(b) is a drawing of two crossing planar electrode arrays, FIG. 1(c) is a drawing of a cylindrical electrode array, FIG. 1(d) is a drawing of two crossing cylindrical electrode arrays, and FIG. 1(e) is a drawing of cylindrical and circular electrode arrays.

FIG. 2 is a graph that shows the velocity field in the plane orthogonal to the electrodes for the electrode configuration shown FIG. 1(c). The velocity field is measured par Particle Image Velocimetry at mid height of a cell measuring 570 μm high, 2 mm wide and 2 mm long. The fluidic solution was water with conductivity α=0.6 S.m⁻¹ seeded with 0.71 μm fluorescent particles. The signal applied was 530V.cm⁻¹ at 700 KHz.

FIG. 3 is a block diagram that illustrates one possible configuration of the arrangement of the electrode arrays.

### DETAILED DESCRIPTION OF THE INVENTION

In the following description of the preferred embodiment, reference is made to the accompanying drawings which form a part hereof, and in which is shown by way of illustration a specific embodiment in which the invention may be practiced. It is to be understood that other embodiments may be utilized and structural changes may be made without departing from the scope of the present invention.

Overview

The impact of the manipulation of fluids and/or particles induced by electric fields is described theoretically and experimentally herein. By means of a microfluidic device comprising a periodic array of microelectrodes, fluid(s) and/or particles manipulations are shown including concentration, separation, transport or mixing using electrokinetic properties. The theoretically predicted dynamical phenomena are demonstrated experimentally.

This invention could be used, for example, to improve the mixing of microliter or nanoliter volume protein solutions analyzed in high throughput screening assays. Electrokinetic micromixing improves the time and reliability for protein expression by rapidly homogenizing the small volume solution. Current methods require extensive human or robotic operations and generally lack the required sensitivity to meet reliability testing standards. Other possible applications could be the separation and detection of small populations of pre-cancerous cells from body fluids (blood, sputum, urine), the concentration of DNA particles inside a Polymerase Chain Reaction (PCR) apparatus for improved DNA detection or enhancement in quality and duration of ELISA assays. Another possible application is cell electroporation. Electroporation, or electropermeabilization, is a significant increase in the electrical conductivity and permeability of the cell plasma membrane caused by an externally applied electrical field. It is usually used in molecular biology as a way of introducing some substance into a cell, such as loading it with a molecular probe, a drug that can change the cell's function, or a piece of coding DNA [28]. The current invention can serve to provide electric fields for electroporation while keeping the cells in suspension by induced fluid flow. This can be done by bringing an external assembly of electrodes in contact with the solution, or by embedding the electrodes inside the well-plate walls, specifically for standard 1536-well plate, 3456-well plate and higher well-plate formats, not excluding the plates with any other number of wells.

Technical Description

The present invention discloses an apparatus and method to achieve manipulation of particles and/or solution in micro- to nanoliter volumes.

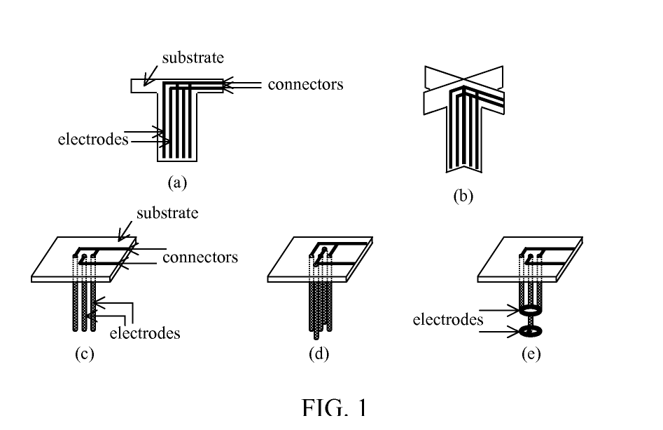

FIGS. 1(a)-1(e) are block diagrams that illustrate some examples of the arrangement of electrode arrays used in an embodiment of the present invention, wherein FIG. 1(a) is a drawing of a planar electrode array, FIG. 1(b) is a drawing of two crossing planar electrode arrays, FIG. 1(c) is a drawing of a cylindrical electrode array, FIG. 1(d) is a drawing of two crossing cylindrical electrode arrays, and FIG. 1(e) is a drawing of cylindrical and circular electrode arrays.

The device of this invention uses electrokinetic properties. The manipulation is performed by bringing the active part of the device (i.e., the electrode arrays) into contact with the fluidic solution considered and applying precise and carefully chosen electric fields combinations.

The purpose of the active part of the device is to manipulate the flow and/or particles using an electric field, including actively changing properties of particles by use of electromagnetic fields.

Electric fields induce a force on charged particles in solutions, moving the particles towards either the cathode or the anode depending on the sign of the charged particles [22]. Such a particle motion in liquid phase is called electrophoresis. If the particle is uncharged, applying AC-electric field to the medium containing the particles creates a dipole on the particles. The orientation of the dipole depends on the conductivity and permittivity of both the particles and the medium. For dielectric particles, the expression of the time average force is given by

[symbol]

F_{DEP} [symbol] =2πa³∈ₘRe[K(Ω)]∇|E|²

where E is the rms electric field, a is the particle radius, Ω is the angular field frequency, and Re[z] indicates the real part of the complex number z. The factor K(Ω) is a measure of the effective polarizability of the particle, known as the Clausius-Mossotti factor, given by

K(Ω)=(∈ₚ*−∈ₘ*)/(∈ₚ*+2∈ₘ*)

where ∈ₚ* and ∈ₘ* are the complex permittivities of the particle and the medium, respectively. The complex permittivity is defined as ∈*=∈−i(σ/Ω), where i=\sqrt{−1}, is the permittivity, and σ is the conductivity.

The particles submitted to a non uniform electric field will move toward or away the high electric field region depending on the sign of Re[K(Ω)]. The motion of the particles is called dielectrophoresis.

Electrophoresis and dielectrophoresis are two major subjects in particle separation and transport. For separation purposes, let's consider a common case where two types of particles are present in the solution.

Separation occurs when there is a frequency Ωₛ for which Re[K(Ω)] takes a different sign for each particle type. For particles having close properties Ωₛ might be impossible to apply experimentally. In that case [23] have shown that two superposed AC-electric fields with two different frequencies Ωₛ₁ and Ωₛ₂ enables the particle separation. Ωₛ₁ and Ωₛ₂ being two frequencies for which each particle type has a Clausius-Mossotti factor of opposite sign.

Consider a simple but commonly used configuration of an electrode array for which a closed-form solution of the electric field and the DEP force was derived in [23]. It is comprised of a periodic array of long parallel micro-electrodes. Each electrode submitted to an AC-electric field with a defined phase difference with their neighbors will simultaneously separate and transport the particles through the system [23]. The process is named traveling wave dielectrophoresis.

Electric fields induce fluid and/or particle motions through several electro-hydrodynamic, electrophoretic or dielectrophoretic effects. Among all the effects the flow is submitted to, the most important in microelectrode devices are electrothermal convection and electroosmosis. The former appears to be due to a non-uniform Joule heating of the fluid which leads to gradients of its permittivity and conductivity. The applied electric fields acting on the permittivity and conductivity gradients generate electrical body forces that induce the flow [13]. The latter is caused by electrical stresses in the diffuse double layer of charges accumulated above the electrodes (AC-electroosmosis) [14] or at the walls (electroosmosis) [24]. These stresses result in a rapidly varying fluid velocity profile in the diffuse double layer, going from zero at the wall to a finite value just outside the double layer. Whether electrothermal or AC-electroosmotic flows dominate the motion of fluid in the device depends mainly on the frequency of the applied electric field and the conductivity of the medium, AC-electroosmosis being dominant at a frequency range several orders of magnitude below the charge relaxation frequency (Ω_{c}≈σ/∈) for low conductivity media.

If the applied frequency is chosen carefully, the induced effects will most affect the fluid flow and produce efficient mixing. Using multifrequency electric field signals [8] can improve the flow and particle manipulation effects.

Dielectrophoresis and fluid flow precisely combined make possible the manipulation of submicron particles [25]. For a careful choice of the applied frequency, the electro-hydrodynamically induced fluid flows but will be determinant in the DEP manipulation and/or separation of submicron particles. It has been shown that the induced dynamical properties can be creatively used as a mechanism to control micro or submicron particles.

Experiments and numerical simulations of the coupled electro-thermo-hydrodynamic problem in devices with interdigitated arrays of electrodes [12, 13, 14] or electrode poles [26] show that both electrothermal and AC-electroosmotic flows consist of convective rolls centered at the electrode edges and provide good estimates for their strength and frequency dependence. Near the electrodes, the fluid velocity u₀ ranges from 1 to 1000 μm.s⁻¹ decaying exponentially with the transversal distance to the electrodes.

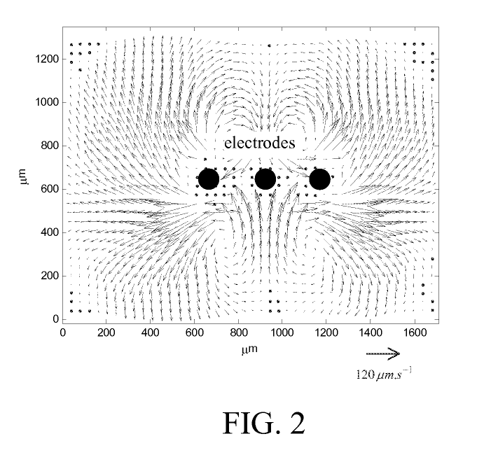

FIG. 2 is a graph that shows the velocity field in the plane orthogonal to the electrodes for the electrode configuration shown FIG. 1(c). The velocity field is measured par Particle Image Velocimetry at mid height of a cell measuring 570 μm high, 2 mm wide and 2 mm long. The fluidic solution was water with conductivity σ=0.6 S.m⁻¹ seeded with 0.71 μm fluorescent particles. The signal applied was 530V.cm⁻¹ at 700 KHz. As shown in FIG. 2, in a device of characteristic length d=150 μm, fluid viscosity v=10⁻⁶ m² s⁻¹, conductivity σ=0.6 S.m⁻¹ with AC-electric field of 530V/cm, the maximum flow velocity is measured to be 150 μm.s⁻¹.

The electric field induced heating inside the solution induces buoyancy flow effects. These are caused by gravity acting on nonhomogeneities in densities inside the liquid solution to induce flow. These are possibly used in the device in conjunction with electrokinetic/electrothermal effects to provide mixing, concentration, separation and transport effects.

The invention apparatus contains electrodes capable of producing any of the physical properties described in the sections above. The device is capable of inducing electrokinetic (including electroosmotic and electrothermal) fluid flow inside vessels (including microplates or well-plates and microarray hybridization solutions). The electrode arrays are designed to fit microliter size (or smaller) vessels as well as microliter (or smaller) droplets. The electrodes are generally micron sized wires shaped like, or deposited layers of metal on a substrate, as shown in FIGS. 1(a)-1(e). When electrodes are positioned inside the vessel, the device can be manufactured by comolding conducting polymer electrodes and a plastic. For micromixing, one might prefer pole electrodes since the fluid can flow between the electrodes, for instance as shown in FIG. 2. The electrode array pitch is optimized depending on the application and the electric field strength.

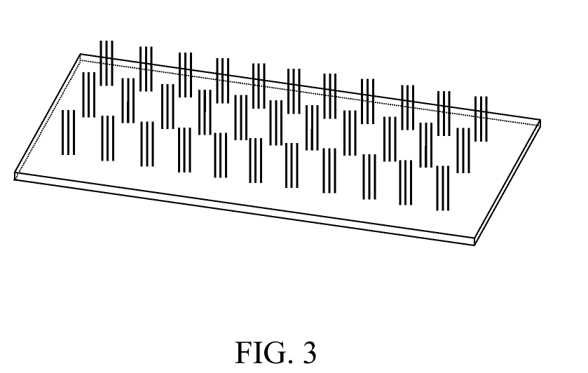

For parallel applications like well-plates or microarrays, the device can comprise sets of individually tunable or not tunable electrode arrays, as shown in FIG. 3, which is a block diagram that illustrates one possible configuration of the arrangement of the electrode arrays.

The device is either applied externally by inserting electrodes inside vessels, or the device is built into the vessel, itself, if flow is generated by forces other than single-frequency electroosmosis. The device can be used for general manipulation of fluids and particles inside the vessel, including concentration, separation, transport, mixing or electroporation. The device is tunable, so that by applying different DC and/or AC voltages, different flow effects can be induced and adapted to efficiently manipulate fluids and particles contained inside the vessel. The device can perform one or more particle manipulation operations. The device is flexible. It can be tuned and adapted to a variety of configurations depending on the application.

The process of this invention is to bring the electrodes in contact with the fluid and/or particle solution. If flow is generated by forces other than single-frequency electroosmosis, the electrodes can be embedded within the container or plate. Inserting the electrodes in the solution and applying the appropriate electric field will enable manipulation of the fluidic solution.

### REFERENCES

The following references are incorporated by reference herein.
1. H. A. Pohl, Dielectrophoresis (Cambridge University Press, 1978).
2. N. G. Green, A. Ramos, A. Gonzalez, H. Morgan, A. Castellanos, Phys. Rev. E 61, 4011 (2000).
3. A. Ramos, H. Morgan, N. G. Green, A. Castellanos, J. Phys. D: Appl. Phys. 31, 2338 (1998).
4. N. G. Green, A. Ramos, A. Gonzalez, H. Morgan, A. Castellanos, Phys. Rev. E 66, 026305 (2002).
5. PETHIG R, HUANG Y, WANG X B, BURT J P H, “POSITIVE AND NEGATIVE DIELECTROPHORETIC COLLECTION OF COLLOIDAL PARTICLES USING INTERDIGITATED CASTELLATED MICROELECTRODES,” Journal of Physics D-Applied Physics 25 (5): 881-888 May 14, 1992.
6. Arnold W M, Franich N R, Cell isolation and growth in electric-field defined micro-wells, APPLIED PHYSICS 6 (3): 371-374 June 2006
7. H. Morgan, M. P. Hughes, and N. G. Green, “Separation of submicron bioparticles by dielectrophoresis,” Biophysical Journal 77 (1999) 516-525.
8. I. Tuval, I. Mezic, F. Bottausci, Y. T. Zhang, N. C MacDonald and O. Piro, Control of particles in microelectrode devices, Physical Review Letters 95(23) December 2005.
9. J. Suchiro, and R. Pethig, “The dielectrophoretic movement and positioning of a biological cell using a three-dimensional grid electrode system,” Journal of Physics D: Applied Physics 31 (1998) 3298-3305.
10. T. Müller, G. Gradl, S. Howitz, S. Shirley, T. Schnell, and G. Fuhr, A 3-D microelectrode system for handling and caging single cells and particles, Biosensors & Bioelectronics 14 (1999) 247-256.
11. Marshall A and Hodgson J 1998 DNA chips: an array of possibilities Nat. Biotechnol. 16 27.
12. Southern E, Mir K and Shchepinov M, 1999 Molecular interactions on microarrays, Nat. Genet. 21 (Suppl.) 5-9.
13. Xiang C C and Chen Y 2000 Biotechnol. Adv. 18 35-46.
14. Liu J, Williams B A, Gwirtz R M, Wold B J, Quake S, ANGEWANDTE CHEMIE-INTERNATIONAL EDITION 45 (22): 3618-3623 2006.
15. Yuen P K, Li G S, Bao Y J, Muller U R, Microfluidic devices for fluidic circulation and mixing improve hybridization signal intensity on DNA arrays LAB ON A CHIP 3 (1): 46-50 2003.
16. Sasakura Y, Kanda K, Fukuzono S, Microarray techniques for more rapid protein quantification: Use of single spot multiplex analysis and a vibration reaction unit, ANALYTICA CHIMICA ACTA 564 (1): 53-58 Mar. 30, 2006.
17. F Fixe, H M Branz, N Louro, V Chul, D M F Prazeres and J P Conde, Electric-field assisted immobilization and hybridization of DNA oligomers on thin-film microchips, Nanotechnology 16 (2005) 2061-2071.
18. Sigurdson M, Wang D Z, Meinhart C D, Electrothermal stirring for heterogeneous immunoassays, LAB ON A CHIP 5 (12): 1366-1373 2005.
19. Robin H. Liu, Ralf Lenigk, Piotr Grodzinski, Acoustic micromixer for enhancement of DNA biochip systems J. Microlith., Microfab., Microsyst., Vol. 2 No. 3, July 2003 179.
20. J Vanderhoeven, K, Pappaert, B, Dutta, P. V, Hummelen and G. Desmet DNA Microarray Enhancement Using a Continuously and Discontinuously Rotating Microchamber, Anal. Chem. 2005, 77, 4474-4480.
21. M. Hartmann a, A. Toeglb, R. Kirchner b, M. F. Templin a, T. O. Joos, Increasing robustness and sensitivity of protein microarrays through microagitation and automation, Analytica Chimica Acta 564 (2006) 66-73.
22. D. Li, electrokinetics in microfluidics, Elsevier Academic Boston 2004.
23. D. E. Chang, S. Loire, I. Mezi'c, J. Phys. D: Appl. Phys. 36, 3073 (2003).
24. Yang R J, Fu L M, Lin Y C, Electroosmotic flow in microchannels, JOURNAL OF COLLOID AND INTERFACE SCIENCE 239 (1): 98-105 Jul. 1, 2001.
25. U.S. Patent Publication No. 2007/0175755 A1, filed Nov. 16, 2007, by I. Mezic et al., entitled “DYNAMIC EQUILIBRIUM SEPARATION, CONCENTRATION, AND MIXING APPARATUS AND METHODS.”
26. Squires T M, Bazant M Z, Induced-charge electro-osmosis, JOURNAL OF FLUID MECHANICS 509: 217-252 Jun. 25, 2004
27. J. Voldman, M. Toner, M. L. Gray, and M. A. Schmidt, Design and analysis of extruded quadrupolar dielectrophoretic traps, Journal of Electrostatics 57 (2003) 69-90
28. Neumann E, Schaefer-Ridder M, Wang Y, Hofschneider P H (1982). “Gene transfer into mouse lyoma cells by electroporation in high electric fields”. EMBO J. 1 (7): 841-5.

### CONCLUSION

In this invention, we claim an external or internal device that can be brought into contact with fluids or complex solutions contained in standard 1536, 3456 well-plates, or plates containing another number of wells, or microarrays—not excluding any other formats for vessel containing fluids and particles—in order to manipulate the said fluids or complex solutions. The device has built in electrodes that can be used to manipulate fluids or solutions by creating electric fields.

In this invention, we claim a method to manipulate fluids or fluidic solutions by creating electric fields with electrodes in direct or indirect contact with fluids or fluidic solutions.

In this invention, we claim a process to manipulate fluids or fluidic solutions by creating electric fields with electrodes in direct or indirect contact with fluids or fluidic solutions. The process implies to bring electrodes in contact with one or more fluids or fluidic solutions. The electrodes are energized, and a force is transmitted to the fluids.

This concludes the description of the preferred embodiment of the present invention. The foregoing description of one or more embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto and the full range of equivalents of the claims appended hereto.

What is claimed is:

1. A device for manipulation of fluid and particles using electrokinetic properties resulting from applied electric fields, comprising:
at least one pair of electrodes, made of electrically conductive materials, for manipulating a fluid and particles using electrokinetic properties resulting from applied electric fields generated by the electrodes; and
wherein the electrodes are inserted into a vessel containing the fluid and particles in order to manipulate the fluid and particles.

2. The device of claim 1, wherein the electrodes are planar, cylindrical or a 3D shape, and the electrodes' distribution is symmetrical or non-symmetrical.

3. The device of claim 1, wherein the device is built of rigid or flexible materials, and the electrodes are isolated from the materials, if needed.

4. The device of claim 1, wherein the electrodes are wires.

5. The device of claim 1, wherein the electrodes are controlled independently or jointly.

6. The device of claim 1, wherein the electrodes are mounted on a plate, and the plate is used as a fluid barrier to seal the vessel where the electrodes are inserted.

7. The device of claim 1, wherein the electrodes are built into the vessel.

8. The device of claim 1, where the electrodes are coated with an insulating layer.

9. The device of claim 1, wherein the fluid is a gas, a gas with at least one organic or inorganic particle, a liquid, or a liquid with at least one organic or inorganic particle.

10. The device of claim 1, wherein the fluid is mixed or dispersed by applying a time-dependent electrohydrodynamic fluid flow using a fluid motivating force, and the fluid motivating force is an electromechanical, mechanical or electrochemical force.

11. The device of claim 1, wherein the particles are concentrated, separated, transported, mixed or dispersed by applying a time-dependent electrohydrodynamic fluid flow together with a particle motivating force.

12. The device of claim 1, wherein the fluid or particles are charged or neutral.

13. The device of claim 1, wherein the particles are detected and collected.

14. The device of claim 1, wherein the vessel is a microtitter plate container.

15. The device of claim 1, wherein the vessel is a microarray plate.

16. The device of claim 1, wherein a polymerase chain reaction (PCR) is performed within the vessel, by energizing the electrodes, in order to perform thermocycling and enhance a readout signal.

17. The device of claim 1, wherein a kinase-based or ELISA assay is performed within the vessel, by energizing the electrodes, such that the concentration needed to obtain a sufficient signal is reduced.

18. The device of claim 1, wherein an electroporation process is performed within the device, by applying an electromagnetic field using the electrodes, in order to simultaneously enable opening of pores in a cell membrane using an electric pulse and to enhance efficient transport of the particles that pass through the pores by cell suspension, mixing or concentration.

19. A method for manipulation of fluid and particles using electrokinetic properties resulting from applied electric fields, comprising:
manipulating a fluid and particles using electrokinetic properties resulting from applied electric fields generated by at least one pair of electrodes made of electrically conductive materials, and the electrodes are inserted into a vessel containing the fluid and particles in order to manipulate the fluid and particles.
